(19) United States
(12) Patent Application Publication
(10) Pub. No.: US 2025/0127900 A1
(43) Pub. Date: Apr. 24, 2025

(54) STABLE PHARMACEUTICAL COMPOSITIONS

(71) Applicant: MANKIND PHARMA LTD., New Delhi (IN)

(72) Inventors: Vivek JAIN, Haryana Gurugram (IN); P. V. S NARASIMHAM, Haryana Gurugram (IN); Chithambaram MUTHULINGAM, Haryana Gurugram (IN); Anil KUMAR, Haryana Gurugram (IN); Amit BANSAL, Haryana Gurugram (IN)

(21) Appl. No.: 18/730,348

(22) PCT Filed: Dec. 17, 2022
(86) PCT No.: PCT/IB2022/062425

§ 371 Date: Jul. 19, 2024

(30) Foreign Application Priority Data
Dec. 22, 2021 (IN) 202111059970

(51) Int. Cl. A61K 47/22; A61K 9/20; A61K 31/14; A61K 47/10

(52) U.S. Cl. CPC A61K 47/22; A61K 9/20; A61K 31/14; A61K 47/10

(57) ABSTRACT

The present invention relates to a stable pharmaceutical composition of therapeutically active agent, wherein the amount of nitrosamine impurity is below the FDA acceptable intake limit for shelf life of the pharmaceutical composition and a process for preparing the same.

### FIELD OF INVENTION

The present invention relates to a stable pharmaceutical composition of therapeutically active agent, wherein the amount of nitrosamine impurity is below the FDA acceptable intake limit for shelf life of the pharmaceutical composition and a process for preparing the same.

### BACKGROUND OF THE INVENTION

Nitrosamines are found in a wide variety of drug products in amounts ranging from trace amounts to fairly large quantities. The formation of N-nitrosamines in drug products apparently takes place by a variety of mechanisms, at least some of which are still unknown.

Many N-nitrosamines are highly toxic. N, N-dimethylnitrosamine, for example, has a LD₅₀ of only 10 mg/Kg. Of greater significance than their toxicity, however, are potential carcinogenic, mutagenic, and teratogenic effects. Many N-nitrosamines, are classified as animal carcinogens.

Because of the aforementioned health-related effects, stringent limitations have been placed upon the permissible amounts of N-nitrosamine impurities in drug products. Because of the exceptionally low allowable limits for N-nitrosamine impurities in these products it become important to control the nitrosamine impurities during synthesis of active pharmaceutical ingredient or drug formulation.

It has been noted that several drugs have been recalled from US market many times because of the presence of the nitrosamine impurities at or above the FDA acceptable intake limit. There are multiple reasons why nitrosamines can be present in drugs. FDA found that the source of nitrosamines can be related to the drug's manufacturing process or its chemical structure or even the conditions in which they are stored or packaged. As foods and drugs are processed in the body, nitrosamines can also be formed. During process of preparing the therapeutically active agent it is possible that trace amounts of nitrosating agents derived from prior nitration processes in nitro-substituted intermediates react subsequently with amines or substituted amines to form the corresponding N-substituted-N-nitrosamines. Nitrosamine impurities may increase the risk of cancer if people are exposed to them above acceptable levels and over longer periods of time.

There is always a need in art to develop a pharmaceutical composition of therapeutically active agents which is stable and the amount of nitrosamines impurities does not exceed beyond the FDA acceptable intake limit for shelf life of the pharmaceutical composition.

### SUMMARY OF THE INVENTION

The present invention relates to a stable pharmaceutical composition comprising a therapeutically active agent, one or more stabilizing agents, and one or more pharmaceutically acceptable excipients.

Another aspect of the present invention provides a stable pharmaceutical composition comprising a therapeutically active agent, one or more antioxidants, and one or more pharmaceutically acceptable excipients.

Another aspect of the present invention provides a stable pharmaceutical composition comprising a therapeutically active agent, one or more antioxidants selected from the group comprising ascorbic acid, tocopherol, gentisic acid (2,5-dihydroxy benzoic acid), butylated hydroxytoluene, butylated hydroxylanisole, propyl gallates, and one or more pharmaceutically acceptable excipients.

Another aspect of the present invention provides a stable pharmaceutical composition comprising a therapeutically active agent, one or more pH modifiers, and one or more pharmaceutically acceptable excipients.

Another aspect of the present invention provides a stable pharmaceutical composition comprising a therapeutically active agent, one or more pH modifiers selected from the group comprising organic acid or inorganic acid selected from ascorbic acid, fumaric acid, citric acid, malic acid, succinic acid, adipic acid, maleic acid, lactic acid, hydrochloric acid, nitric acid, phosphoric acid, sulfuric acid; organic or inorganic base selected pyridine, alkanamines, methylamine, diisopropylamine, diisopropylethylamine triethylamine, dimethylamine, trimethyl amine, pyridine, imidazole, histidine, guanidine, poly ethyleneimine, poly(vinylpyridine), diethanolamine, triethanolamine, tris(hydroxymethyl)aminomethane (Tris), sodium glycine, imidazole, 1-methylimidazole, 2-methylimidazole, and 4 (5)-methylimidazole, and 1,2-diaminoethane, 2-(bis(2-hydroxyethyl)amino)-2-(hydroxymethyl) propane-1,3-diol, sodium glycine, sodium lysine, sodium histidine, and sodium arginine, polyvinyl imidazole and copolymers thereof, sodium carbonate, sodium bicarbonate, potassium bicarbonate, lithium carbonate, potassium carbonate, calcium carbonate, magnesium carbonate, magnesium oxide, sodium citrate, sodium hydroxide, potassium hydroxide, ammonium salts or a combination thereof, and one or more pharmaceutically acceptable excipients.

The present invention further relates to a process for preparation of a stable pharmaceutical composition comprising a therapeutically active agent, wherein the process comprises: (a) preparing blend or granules of therapeutically active agent, one or more stabilizing agents, and optionally one or more pharmaceutically acceptable excipients; (b) compressing the blend or granules to form tablets or filling the granules in capsules.

The present invention further relates to a process for preparation of a stable pharmaceutical composition comprising a therapeutically active agent, wherein the process comprises: (a) preparing blend or granules of therapeutically active agent, and one or more pharmaceutically acceptable excipients; (b) adding one or more stabilizing agents to the blend or granules to obtain a final blend; c) compressing the final blend to form tablets or filling the granules in capsules.

The present invention further relates to a process for preparation of a stable pharmaceutical composition comprising a therapeutically active agent, wherein the process comprises: (a) preparing suspension(s) or solution(s) of the therapeutically active agent, one or more stabilizing agents, and one or more pharmaceutically acceptable excipients in a solvent(s) together or separately; (b) adding the suspension(s) or solution(s) of step (a) onto one or more pharmaceutical acceptable excipients to make granules; (c) compressing the granules to form tablets or filling the granules in capsules.

### DESCRIPTION OF THE INVENTION

The present invention relates to a stable pharmaceutical composition comprising a therapeutically active agent, one or more stabilizing agents, and one or more pharmaceutically acceptable excipients.

As used in this specification, the singular forms “a”, “an”, and “the” include plural references unless the context clearly dictates otherwise. Thus for example, a reference to “a method” or “a process” includes one or more methods, one or more processes and/or steps of the type described herein and/or which will become apparent to those persons skilled in the art upon reading this disclosure and so forth.

The term “pharmaceutical composition” or “composition” or “solid oral composition” or “dosage form” or “drug product” as used herein synonymously include tablet such as mono-layered tablets, bilayered tablets, trilayered tablet, multilayer tablet, caplets, minitablets, capsules, tablet in tablet, tablets in a capsule, granules in a capsule, pellets, pellets in a capsule, powder, granules, suspension, solution, cream, gel, or any other suitable dosage form meant for oral, parenteral, topical, or transdermal administration including ophthalmic, otic, inhalation. The pharmaceutical composition includes immediate release, fast release, orally disintegrating, extended release, modified release, delayed release, pulsed release compositions and the like.

The term “stable pharmaceutical composition” according to present invention refers to a pharmaceutical composition comprising therapeutically active agent(s) in which the amount of nitrosamine impurity remains below FDA acceptable intake limit after exposure of the pharmaceutical composition to 40° C./75% RH for a period of six months or 25° C./60% RH for a period of at least 12 months.

The terms “FDA acceptable intake limit” of nitrosamine impurity as used in the present invention is the upper limit set by FDA for daily intake of nitrosamine impurity below which there is no harmful side-effect likely to occur on long term treatment with the therapeutically active agent. In accordance with the present invention, the nitrosamine impurity is reduced to less than about 50 ppm, preferably less than about 25 ppm, more preferably less than about 5 ppm. The nitrosamine impurity is reduced to less than FDA acceptable intake limit of the nitrosamine impurity set by FDA for a specific therapeutically active agent based on its maximum daily dose.

Daily intake limit of nitrosamine impurity depends upon the chemical structure of a particular impurity, as different impurities have different pharmacological behaviour and accordingly different daily intake limit as exemplified in Table 1 below:

TABLE 1
Acceptable intakes for specific nitrosamine impurities

| | | EMA | FDA |
|---|---|---|---|
| Impurity | Code | (ng/day) | (ng/day) |
| N-Nitrosodimethylamine | NDMA | 96 | 96 |
| N-Nitrosodiethylamine | NDEA | 26.5 | 26.5 |
| N-Nitrosodiisopropylamine | NDIPA | 26.5 | 26.5 |
| N-Nitrosomethylphenylamine | NMPA | 34.3 | 26.5 |

The therapeutically active agent(s) is a compound or agent that has biologic activity in vivo and is susceptible to degradation to nitrosamine impurities. The therapeutically active agent may be selected from but not limited to anti-depressant agents such as paroxetine, fluoxetine, sertraline, citalopram, escitalopram, venlafaxine, desvenlafaxine, varenicline, and mirtazapine, anti-epileptic agents such as carbamazepine, oxcarbazepine, gabapentin, pregabalin and tiagabine, antihypertensive agents such as ramipril, quinapril, enalapril, perindopril, trandolapril, captopril, lisinopril, oxeprenolol, nifedipine, nevibolol, atenolol, verapamil, hydralazine, pindolol, metoprolol, metformin, carvedilol, bisoprolol, diltiazem, furosemide and propranolol, proton pump inhibitors such as omeprazole, lansoprazole, esomeprazole, rabeprazole and pantoprazole, angiotensin type II receptor antagonists such as candesartan, eprosartan, irbesartan, losartan, telmisartan, olmesartan, azilsartan, and valsartan, anti-diabetic agents such as repaglinide and the glitazones (troglitazone, ciglitazone, pioglitazone and rosiglitazone), glipizide, sitagliptin, vildagliptin, saxagliptin, NVP DPP728, P32/98, FE 15 999011, PHX1149, dapagliflozin, empagliflozin, anti-schizophrenic agents such as aripiprazole, thioridazine, chlorpromazine, clozapine, zuclopenthixol, flupenthixol, droperidol, haloperidol, risperidine, quetiapine, amisulpride and olanzapine agents for treating ADHD such as methylphenidate and atomoxetine, and anti-cholesteremia agents such as gemfibrozil, colestipol, ezetemibe, fluvastatin, simvastatin, fenofibrate, atorvastatin and pravastatin, 20 diuretics such as furosemide, bumetanide, hydrochlorthiazide, spironolactone, or pharmaceutically acceptable salts thereof, malarial treatment agents such atovaquone and proguanil or pharmaceutically acceptable salts thereof, other therapeutically active agents baclofen, succinylcholine, suggamadex, timolol, tobramycin, carbidopa, levodopa, phenytoin, oxybutynin, Phytonadione, Pimavanserin, Prazosin, Prochlorperazine, Polymyxin, Tetracaine, Tranexamic, acid, Glyceryl Trinitrate, Trimethoprim, Silodosin, Rosuvastatin, Sacubitril nitrofurantoin, haloperidol, divalproex, nitroglycerine, levothyroxine, rivaroxaban, apixaban, updacitinib, ticagrelor, clozapine, mesalamine, midodrine, diltiazem, difluprednate, loteprednol, formoterol, arformoterol, albuterol, levoalbuterol, vasopressin, phenylephrine, lidocaine, brimonidine, bimatoprost, tobramycin, brinzolamide, dorzolamide, tetracaine, ephedrine, epinephrine, norepinephrine, moxifloxacin, ofloxacin, ciprofloxacin, amoxicillin, azithromycin, Revefenacin, rocuronium, cistracurium, predinisolone, fluorometholone, budesonide, desmopressin, betamethasone, latanoprost, dexamethasone, hydrocortisone, Neomycin, liftegrat, Naloxone, dydrogestrone, liothryonine, methylprednisolone, glycopyrrolate, proparacaine, acetazolamide, alosteron, Amitriptylinetorvastatin, Apremilast, Aripiprazole, Doxepin, Elagolix, Atropine, brivaracetam, chlorthalidone, nitroglycerin, clomipramine, Famotidine, Fenofibrate, or their pharmaceutical acceptable salts and combinations thereof.

The therapeutically active agent(s) can exist as a free base or as its pharmaceutically acceptable salt. The term “pharmaceutically acceptable salt” or “salts” as used in the context of the present invention refers to inorganic acids salts or organic acids salts and the like.

The term “stabilizing agent(s)” as used in present invention is any suitable pharmaceutically acceptable excipient capable of keeping the amount of nitrosamine impurity less than about 50 ppm, preferably less than about 25 ppm, more preferably less than about 5 ppm, in particular less than the FDA acceptable intake limit and is selected from the group comprising of antioxidants, amino acids, sugar alcohols, carbohydrates, pH modifiers, or a mixture thereof.

Antioxidant is selected from but not limited to, ascorbic acid, tartaric acid, succinic acid, maleic acid, fumaric acid, citric acid, malic acid, lactic acid, benzenesulfonic acid, oxalic acid, triphenylacetic acid, 1-hydroxy-2-naphthoic acid, 3-hydroxy-2-naphthoic acid, sodium ascorbate, alpha-tocopherol, butylated hydroxytoluene, butylated hydroxyanisole, gentisic acid (2,5-dihydroxy benzoic acid), acetyl cysteine, ascorbyl palmitate, cysteine, dithiothreitol, thioglycerol, thiourea, caffeic acid, propyl gallate, ferulic acid, sodium pyrosulfite, edetic acid, edetate salts, 2, 6-di-tert-butyl p-cresol, gallic acid and esters thereof, nordihydroguaiaretic acid, guaiacol ester, tea polyphenol, curcumin, chlorogenic acid, methionine, proline, biflavonoids, superoxide dismutase, silymarin, grape skin/seed extract, melanin, rosemary extract, sodium sulfite, sodium thiosulfate, sodium bisulfite, sodium metabisulfite, tert-butylhydroquinone, thiourea, methionine, sodium citrate, magnesium ascorbyl phosphate, sodium ascorbyl phosphate, 3,4-dihydroxybenzoic acid, butylated hydroxybenzoic acid and salts thereof, erythorbic acid and sodium salts thereof, sorbic acid and salts thereof, sodium formaldehyde sulfoxylate, glutathione, lipoic acid, dihydroxy fumaric acid, and the like.

Amino acid is selected from but not limited to glycine, lysine, histidine, leucine, arginine, alanine, asparagine, cysteine, glutamine, isoleucine, methionine, phenylalanine, proline, serine, threonine, tryptophan, tyrosine, valine, and the like.

Sugar alcohol is selected from but not limited to alditols, polyols, mannitol, meglumine, xylitol, isomalt, sorbitol, lactitol, pentitol, arabitol, ribitol, galactitol, erythritol, glycerol, and the like.

Carbohydrate is selected from but not limited to monosaccharides, oligosaccharides, polysaccharides, dextrins, maltodextrin, pullulan, arabinose, dextrose, lactose, sucrose, sucralose, saccharin, fructose, maltose, trehalose, psicose, tagatose, sorbose, cellulose derivatives, cellobiose, starches, sucrose fatty acid esters, and the like.

pH modifier is selected from an acid selected from an organic or inorganic acid such as ascorbic acid, fumaric acid, citric acid, malic acid, succinic acid, adipic acid, maleic acid, lactic acid, hydrochloric acid, nitric acid, phosphoric acid, sulfuric acid, and the like or a base selected from organic bases such as pyridine, alkanamines, such as methylamine, diisopropylamine, diisopropylethylamine triethylamine, dimethylamine, trimethyl amine, pyridine, imidazole, histidine, guanidine, ethyleneimine, poly poly(vinylpyridine), diethanolamine, triethanolamine, tris(hydroxymethyl)aminomethane (Tris), sodium glycine, imidazole, 1-methylimidazole, 2-methylimidazole, and 4 (5)-methylimidazole, and 1,2-diaminoethane, 2-(bis(2-hydroxyethyl)amino)-2-(hydroxymethyl) propane-1,3-diol, sodium glycine, sodium lysine, sodium histidine, and sodium arginine, polyvinyl imidazole, and copolymers thereof (e.g., a copolymer of poly ethyleneimine and one or more of poly(vinylpyridine) and polyvinylimidazole, or a copolymer of poly(vinylpyridine) with polyvinyl imidazole) or inorganic bases such as sodium carbonate, sodium bicarbonate, potassium bicarbonate, lithium carbonate, potassium carbonate, calcium carbonate, magnesium carbonate, magnesium oxide, sodium citrate, sodium hydroxide, potassium hydroxide, ammonium salts, and the like

According to one of the embodiment, therapeutically active agent and stabilizing agent(s) are present in the composition in a ratio of 1:0.01 to 1:25.

Another embodiment of the present invention provides a stable pharmaceutical composition comprising a therapeutically active agent, antioxidant, and one or more pharmaceutically acceptable excipients.

According to one of the embodiment, therapeutically active agent and antioxidant(s) are present in the composition in a ratio of 1:0.01 to 1:25.

Another embodiment of the present invention provides a stable pharmaceutical composition comprising a therapeutically active agent, ascorbic acid, and one or more pharmaceutically acceptable excipients.

Another embodiment of the present invention provides a stable pharmaceutical composition comprising a therapeutically active agent, butylated hydroxytoluene, optionally butylated hydroxyanisole, and one or more pharmaceutically acceptable excipients.

Another embodiment of the present invention provides a stable pharmaceutical composition comprising a therapeutically active agent, butylated hydroxyanisole, and one or more pharmaceutically acceptable excipients.

Another embodiment of the present invention provides a stable pharmaceutical composition comprising a therapeutically active agent, magnesium oxide, and one or more pharmaceutically acceptable excipients.

The term “excipient(s)” or “pharmaceutically acceptable excipient(s)” means a component of a pharmaceutical product that is not an active ingredient, and includes but not limited to fillers, diluents, disintegrants, glidants, lubricants, surface active agents, buffers, isotonicity agents, viscosity enhancers, pH adjusting agents, preservatives, vehicles, bulking agents and lyoprotectants and/or combinations thereof. The excipients may also include excipients for providing fast release, immediate release, extended release, delayed release, pulsed release, orally disintegration and the like. The excipients that are useful in preparing a pharmaceutical composition are generally safe, non-toxic, and neither biologically nor otherwise undesirable, and are acceptable for veterinary use as well as human pharmaceutical use. One excipient can perform more than one function.

Suitable fillers/diluents include, without limitation, starch, corn starch, potato starch, pregelatinized starch, dry starch, disaccharides, lactose, cellulose, maltodextrins, cellulose derivatives, such as silicified microcrystalline cellulose, microcrystalline cellulose (e.g., cellulose MK GR), mannitol, sorbitol, xylitol, trehalose, colloidal silica, sucrose or other sugars or sugar derivatives, calcium hydrogen phosphate, dicalcium phosphate, low-substituted hydroxypropyl cellulose, hydroxyethyl cellulose, hydroxypropyl methyl cellulose, and combinations thereof. When present, a filler may be employed in an amount ranging from about 10% to about 80%, preferably from about 20% to about 80% by weight of the pharmaceutical composition.

Suitable binders include, without limitation, microcrystalline cellulose, polyvinylpyrrolidone (PVP), e.g., PVP K 30 or PVP90F, polyethylene glycols (PEG), e.g., PEG 4000, hydroxypropylmethyl cellulose, hydroxypropyl cellulose, both preferably of medium to high viscosity, e.g. viscosity grades 3 or 6 cps, copovidone, maltodextrins, pregelatinized starch and combinations thereof. When present, a binder may be employed in an amount ranging from about 0.1% to about 20%, by weight of the pharmaceutical composition.

Suitable lubricants include, without limitation, zinc stearate, magnesium stearate, sodium stearyl fumarate, calcium stearate, stearic acid, colloidal silica, sodium lauryl sulfate, aluminum or calcium silicate, stearic acid, cutina, PEG 4000-8000, talc and combinations thereof. When present, a lubricant may be employed in an amount ranging from about 0.01% to about 10%, preferably from about 0.1% to about 5%, by weight of the pharmaceutical composition,

Suitable glidants include, without limitation, colloidal silicon dioxide (e.g., Aerosil 200), magnesium trisilicate, powdered cellulose, starch, talc and combinations thereof. When present, a glidant may be employed in an amount ranging from about 0.01% to about 10%, preferably from about 0.1% to about 5%, by weight of the pharmaceutical composition.

Suitable disintegrants include, without limitation, carboxymethylcellulose calcium (CMC-Ca), carboxymethylcellulose sodium (CMC-Na), crosslinked PVP (e.g. crospovidone, polyplasdone XL or kollidon CL), croscarmellose sodium, sodium starch glycolate, polacrillin potassium, low substituted hydroxypropyl cellulose, alginic acid, sodium alginate and guar gum, most preferably crosslinked PVP (crospovidone), crosslinked CMC (Ac-Di-Sol), carboxymethyl starch-Na (pirimojel and explotab) and/or combinations thereof. A disintegrant is employed in an amount of 0.01 to 15%, such as of 0.05 to 12%, such as at least 0.1 to 10%, by weight of the pharmaceutical composition.

The surfactants include but are not limited to anionic, cationic, non-ionic or amphoteric surfactants or those known to the person skilled in the art. Suitable surface active are poloxamer 188, polysorbate 80, Cremophore, Soluplus, lecithin and sodium lauryl sulfate.

The buffers include but are not limited to citrate, phosphate, tris HCl, acetic acid, sodium acetate, amino acids such as glycine, aspartate/aspartic acid, histidine, cysteine, tyrosine, phenylalanine, proline, arginine, threonine, serine, valine, isoleucine, lysine and glutamine.

The isotonicity agents include but are not limited to include dextrose, glycerol, sodium chloride, potassium chloride, glycerine, and mannitol.

The viscosity enhancers include but are not limited to xanthan gum, gellan gum carbomer homopolymer, hydroxypropyl cellulose, hydroxy ethyl cellulose, hydroxypropyl methyl cellulose, carboxyvinyl polymers, polymerized gelatin, alginates and the like

The pH adjusting agents include but are not limited to acids such as include hydrochloric acid, sodium hydroxide, and succinic acid.

The preservatives include but are not limited to benzyl alcohol, parabens (methyl, propyl, butyl), benzalkonium chloride, chlorobutanol, and thiomersal.

The lyoprotectants include but are not limited to sucrose, trehalose, mannitol, glycine, lysine and dextran.

The bulking agents include but are not limited to sucrose, lactose, trehalose, mannitol, sorbitol, glucose, PVP, and hydroxyethyl starch.

Suitable extended release or delayed release excipients, without limitation includes hydrophilic or hydrophobic agents comprise one or more of polyvinyl acetate, cellulose acetate, cellulose acetate butyrate, cellulose acetate propionate, ethyl cellulose, a fatty acid, a fatty acid ester, an alkyl alcohol, a wax, xanthan gum, gellan gum, shellac, rosin, zein (prolamine from corn), povidone, kollidon SR (polyvinyl acetate and povidone), a poly(meth)acrylate, poly(ethylene oxide), polyuronic acid salts, cellulose ethers, xanthan gum, tragacanth gum, gum karaya, guar gum, acacia, gellan gum locust bean gum, alkali metal salts of alginic acid or pectic acid, sodium alginate, potassium alginate, ammonium alginate, polyethylene oxide, carbomer homopolymer, hydroxypropyl cellulose, hydroxy ethyl cellulose, hydroxypropyl methyl cellulose, carboxyvinyl polymers, polymerized gelatin, shellac, methacrylic acid copolymer type C NF, cellulose butyrate phthalate, cellulose hydrogen phthalate, cellulose propionate phthalate, polyvinyl acetate phthalate (PVAP), cellulose acetate phthalate (CAP), cellulose acetate trimellitate (CAT), hydroxypropyl methylcellulose phthalate, hydrogenated castor oil, stearic acid, hydrogenated vegetable oil, glyceryl behenate, glyceryl monostearate, hydroxypropyl methylcellulose acetate, dioxypropyl methylcellulose succinate, carboxymethyl ethyl cellulose (CMEC), hydroxypropyl methylcellulose acetate succinate (HPMCAS), and acrylic acid polymers and copolymers like methyl acrylate, ethyl acrylate, methyl methacrylate and/or ethyl methacrylate with copolymers of acrylic and methacrylic acid esters (Eudragit NE, Eudragit RL, Eudragit RS) and the like. Polymer may be used from 0.1-50% by weight of the composition, preferably 10-50% by weight of the composition.

Suitable coating agent according to present invention may be selected from immediate release, extended release, or delayed release coatings but not limited to, Shellac, cellulose acetate phthalate (CAP), polyvinylacetate phthalate (PVAP), hyroxylpropyl cellulose, hyroxypropyl methylcellulose (HPMC), acrylates, phthalates, and Zein (a corn protein derivative), Hydroxyproply methyl cellulose, Methyl hydroxyethyl cellulose, Ethylcellulose, Povidone, Opadry, and the like.

According to one embodiment of the present invention the stable pharmaceutical composition of the present invention is a stable amorphous solid dispersion of therapeutically active agent, one or more stabilizing agents, and optionally one or more pharmaceutically acceptable excipients.

“Solid dispersion” as used herein refers to the dispersion of therapeutically active agent, where the therapeutically active agent exists in solubilized or amorphous state. The increase in solubility of the drug in solid dispersion is mainly because drug remains in amorphous form which is associated with a higher energy state as compared to crystalline counterpart and due to that it requires very less external energy to dissolve. In order to arrive at such a solid dispersion, it is required to have a substantial amount of API dissolved in a suitable solvent at least at one-time point during preparation of the pharmaceutical composition.

The present invention further relates to a process for preparation of a stable pharmaceutical composition comprising a therapeutically active agent, wherein the process comprises: (a) preparing blend or granules of therapeutically active agent, one or more stabilizing agents, and optionally one or more pharmaceutically acceptable excipients; (b) compressing the blend or granules to form tablets or filling the granules in capsules.

The present invention further relates to a process for preparation of a stable pharmaceutical composition comprising a therapeutically active agent, wherein the process comprises: (a) preparing blend or granules of therapeutically active agent, and one or more pharmaceutically acceptable excipients; (b) adding one or more stabilizing agents to the blend or granules to obtain a final blend; c) compressing the final blend to form tablets or filling the granules in capsules.

The present invention further relates to a process for preparation of a stable pharmaceutical composition comprising a therapeutically active agent, wherein the process comprises: (a) preparing a suspension or solution of the therapeutically active agent, one or more stabilizing agents, and one or more pharmaceutically acceptable excipients in a solvent; (b) adding the suspension or solution of step (a) onto one or more pharmaceutical acceptable excipients to make granules; (c) compressing the granules to form tablets or filling the granules in capsules.

Suitable solvent used for preparing solid dispersion of therapeutically active agent is selected from, but not limited to, the group comprising of alcohol such as methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, t-butyl alcohol, 2-ethoxyethanol, diethylene glycol, polyethylene glycol, 1-, 2-, or 3-pentanol, neo-pentyl alcohol, t-pentyl alcohol, diethylene glycol monomethyl ether, diethylene glycol monoethyl ether, cyclohexanol, phenol, glycerol and the like; halogenated solvent such as dichloromethane, chlorobenzene, tetrachloromethane, 1,2-dichloroethane, trichloroethylene, perchloroethylene, 1,1,1-trichloroethane, 1,1,2-trichloroethane, chloroform and the like; ketone such as acetone, methyl ethyl ketone, methyl isobutyl ketone, diethyl ketone, methyl t-butyl ketone and the like; ester solvent such as methyl acetate, ethyl acetate, isopropyl acetate, n-propyl acetate, propenyl acetate, t-butyl acetate, isobutyl acetate, n-butyl acetate, ethyl formate, methyl acetate, methyl propanoate, ethyl propanoate, methyl butanoate, ethyl butanoate and the like; hydrocarbon solvent such as toluene, xylene, heptane, cyvlohexane and the like, ether such as tetrahydrofuran, methyl tetrahydrofuran, dioxane, diethyl ether, diisopropyl ether, methyl t-butyl ether, glyme, diglyme, dibutyl ether, dimethylfuran, 2-methoxyethanol, 2-ethoxyethanol, anisole and the like; nitrile such as acetonitrile, propionitrile, butanenitrile and the like; water; and a combination thereof.

According to one embodiment of the present invention is provided a process for preparation of a stable pharmaceutical composition comprising a therapeutically active agent, wherein the process comprises: (a) preparing a suspension or solution of one or more stabilizing agents, and optionally one or more pharmaceutically acceptable excipients in a solvent(s); (b) adding the suspension or solution of step (a) onto therapeutically active agent, and one or more pharmaceutical acceptable excipients to make granules; (c) compressing the granules to form tablets or filling the granules in capsules.

According to other embodiment, the therapeutically active agent is spray dried with one or more stabilizing agents.

The pharmaceutical composition of present invention can be formulated by any suitable granulation methods known in the art such as wet granulation, direct compression, dry granulation, or melt granulation.

The present invention further relates to a process for preparation of a stable pharmaceutical composition comprising a therapeutically active agent, wherein the process comprises inert gas purging to reduce the oxygen concentration or substitution with oxygen-free gas or bubbling with oxygen-free gas.

The present invention further relates to a package or storage container for packaging or storing the pharmaceutical composition, wherein the package or container is made up of material that blocks oxygen or is impervious to oxygen or comprises an oxygen scavenger.

The present invention is further illustrated by the following examples which are provided merely to be exemplary of the invention and don't limit the scope of the invention. Certain modifications and equivalents will be apparent to those skilled in the art and are intended to be included within the scope of the present invention.

### EXAMPLES

### Example 1: Pharmaceutical Composition of Propranolol with Ascorbic Acid

| Ingredient | Quantity (mg) |
|---|---|
| Propranolol Hydrochloride | 10.00 |
| Lactose anhydrous | 65.65 |
| Ascorbic Acid | 6.25 |
| Microcrystalline cellulose | 22.50 |
| Croscarmellose sodium | 3.37 |
| Colloidal silicon dioxide | 1.12 |
| Magnesium stearate | 1.12 |
| Stearic acid | 2.25 |

### Process:

a) Propranolol hydrochloride, ascorbic acid, lactose anhydrous, microcrystalline cellulose, croscarmellose sodium, colloidal silicon dioxide and magnesium stearate were sifted and blended.
b) Blended material of step a) was compacted using roller compacter to form granules.
c) Lactose anhydrous, Croscarmellose sodium, colloidal silicon dioxide, stearic acid were sifted and blended to form extragranular material.
d) Granules obtained in step b) were blended with extragranular material of step c) and finally lubricated with magnesium stearate.
e) Granular blend of step d) was compressed to form tablets or filled in capsules.

### Example 2: Pharmaceutical Composition of Propranolol with Ascorbic Acid

| | |
|---|---|
| Ingredient | Quantity (mg) |

Pre-roller compaction blending

| | | | | | |
|---|---|---|---|---|---|
| Propranolol | 10 | 20 | 40 | 60 | 80 |
| Hydrochloride | | | | | |
| Butylated | 0.125 | 0.125 | 0.250 | 0.250 | 0.333 |
| Hydroxyanisole | | | | | |
| Isopropyl Alcohol | q.s | q.s | q.s | q.s | q.s |
| Ascorbic acid | 9.3 | 9.3 | 18.6 | 18.6 | 24.8 |
| Lactose anhydrous | 51.150 | 41.150 | 82.300 | 62.000 | 82.667 |
| Microcrystalline | 22.5 | 22.5 | 45.0 | 45.0 | 60.0 |
| cellulose | | | | | |
| Croscarmellose sodium | 2.250 | 2.250 | 4.5 | 4.5 | 6.00 |
| Colloidal Silicon | 0.375 | 0.375 | 0.750 | 0.750 | 1.00 |
| dioxide | | | | | |
| Lake Blend Orange | 0.300 | 0.300 | 0.600 | 0.900 | 1.200 |

Pre-roller compaction lubrication

| | | | | | |
|---|---|---|---|---|---|
| Magnesium stearate | 0.375 | 0.375 | 0.750 | 0.750 | 1.00 |

Pre lubrication (after compaction)

| | | | | | |
|---|---|---|---|---|---|
| Lactose Anhydrous | 11.250 | 11.250 | 22.50 | 22.50 | 30.00 |
| Croscarmellose sodium | 1.125 | 1.125 | 2.250 | 2.250 | 3.00 |
| Colloidal Silicon | 0.750 | 0.750 | 1.500 | 1.500 | 2.00 |
| Dioxide | | | | | |

Lubrication (After compaction)

| | | | | | |
|---|---|---|---|---|---|
| Stearic Acid | 2.250 | 2.250 | 4.500 | 4.500 | 6.00 |
| Magnesium Stearate | 0.750 | 0.750 | 1.500 | 1.500 | 2.00 |

### Process:

### Pre-Roller Compaction Blending and Lubrication

(a) Solution of butylated hydroxyanisole and butylated hydroxytoluene was prepared using isopropyl alcohol under continuous stirring.
(b) Sifted Propranolol Hydrochloride and loaded in rapid mixing granulator followed by addition of above prepared butylated hydroxyanisole dispersion, and dried in a tray dryer.
(c) Propranolol hydrochloride of step (b), ascorbic acid, lactose anhydrous, microcrystalline cellulose, croscarmellose sodium, colloidal silicon dioxide and lake blend orange colour were sifted and blended.
(d) Sifted Magnesium stearate through suitable sieve and mixed with blend of step (c) in blender.
(e) Blended material of step (d) was compacted using roller compacter to form granules.
   Pre Lubrication and Lubrication (after Compaction)
(a) Lactose anhydrous, Croscarmellose sodium and Colloidal silicon dioxide were sifted and blended.
(b) Stearic acid and magnesium stearate were sifted and mixed with blend of step (a) and the blend thus obtained was compressed.

The pharmaceutical compositions of the present invention as prepared according to example 2 was compared with the marketed product for the amount of N-Nitrosopropranolol and the results are represented as below:

Propranolol Tablets

| | Reference Product | Composition of present |
|---|---|---|
| Strength | (Impax) | invention |
| 10 mg | 24.1 | 0.011 |
| 20 mg | 3.70 | 0.011 |
| 40 mg | 3.50 | 0.012 |
| 80 mg | 4.40 | 0.013 |
| 60 mg | 0.47 | 0.013 |

### Example 3: Pharmaceutical Composition of Varenicline with Butylated hydroxytoluene, Butylated hydroxyanisole and Maltodextrin

| Ingredient | Quantity % w/w |
|---|---|
| Varenicline Tartrate | 0.814 |
| Maltodextrin | 8.143 |
| Butylated hydroxytoluene | 0.357 |
| Butylated hydroxyanisole | 1.904 |
| Microcrystalline Cellulose | 44.497 |
| Anhydrous dibasic calcium phosphate | 28.571 |
| Croscarmellose sodium | 9.524 |
| Colloidal silicon dioxide | 0.476 |
| Magnesium stearate | 0.952 |
| Opadry | 4.762 |

### Process:

(a) Croscarmellose sodium and microcrystalline cellulose and other ingredients were dispensed and sifted.
(b) Solution of butylated hydroxyanisole and butylated hydroxytoluene was prepared using isopropyl alcohol under continuous stirring.
(c) Maltodextrin was dissolved in purified water under continuous stirring. Varenicline Tartrate was added to the solution of maltodextrin in water and dissolved under continuous stirring to form solution of Varenicline Tartrate with maltodextrin in water.
(d) Blend of step (a) was granulated using solution of step (b) and (c) followed by blending with croscarmellose sodium & microcrystalline cellulose.
(e) Blend of step (d) was lubricated using stearic acid.
(f) Lubricated blend of step (e) was compressed using suitable punches and dies to form tablets.
(g) Tablets of step (f) were coated with coating solution.

### Example 4: Pharmaceutical Composition of Varenicline with Butylated hydroxytoluene, Butylated hydroxyanisole, and Hydroxypropyl cellulose

| Ingredient | Quantity % w/w |
|---|---|
| Varenicline Tartrate | 0.814 |
| Hydroxypropyl cellulose | 8.143 |
| Butylated hydroxytoluene | 0.357 |
| Butylated hydroxyanisole | 1.904 |
| Microcrystalline Cellulose | 44.497 |
| Anhydrous dibasic calcium phosphate | 28.575 |
| Croscarmellose sodium | 9.520 |
| Colloidal Silicon Dioxide | 0.476 |
| Magnesium stearate | 0.952 |
| Opadry coat | 4.762 |

### Process:

(a) Microcrystalline cellulose, anhydrous dibasic calcium phosphate, and other ingredients were dispensed and sifted.
(b) Solution of butylated hydroxyanisole and butylated hydroxytoluene was prepared using isopropyl alcohol under continuous stirring.
(c) Hydroxypropyl cellulose was dissolved in purified water under continuous stirring. Varenicline Tartrate was added to the solution of Hydroxypropyl cellulose in water and dissolved under continuous stirring to form solution of Varenicline Tartrate.
(d) Blend of step (a) was granulated using the solution of step (b) and (c) followed by blending the granules together with Croscarmellose sodium, Microcrystalline cellulose and Colloidal silicon dioxide.
(e) Granules of step (d) were lubricated with Magnesium stearate.
(f) Lubricated blend of step (e) was compressed using suitable punches and dies to form tablets.
(g) Tablets of step (f) were coated with coating solution.

### Example 5: Pharmaceutical Composition of Bumetanide with Butylated hydroxytoluene and Butylated hydroxyanisole

| Ingredient | Quantity % w/w |
|---|---|
| Bumetanide | 0.588 |
| Lactose monohydrate | 62.258 |
| Butylated hydroxytoluene | 0.550 |
| Butylated hydroxyanisole | 2.504 |
| Microcrystalline Cellulose | 23.53 |
| Pregelatinized Starch | 8.470 |
| Talc | 1.050 |
| Magnesium stearate | 1.050 |

### Process:

(a) Dispensed and co-sifted Lactose monohydrate & microcrystalline cellulose.
(b) Dispensed and co-sifted Bumetanide and Pregelatinized Starch.
(c) Blended the materials of step (a) and (b).
(d) Prepared the solution of butylated hydroxyanisole and butylated hydroxytoluene using isopropyl alcohol under continuous stirring.
(e) Granulated the blend of step (c) using solution of step (d).
(f) Lubricated the granules of step (e) using talc and magnesium stearate.
(g) Compressed the blend of step (f) using suitable punches and dies to form tablets.

### Example 6: Pharmaceutical Composition of Bumetanide with Magnesium Oxide

| Ingredient | Quantity (mg) | | |
|---|---|---|---|
| Bumetanide | 0.500 | 1.000 | 2.000 |
| Lactose monohydrate | 42.887 | 85.775 | 171.550 |
| Microcrystalline cellulose | 21.250 | 42.500 | 85.000 |
| Pregelatinized Starch | 6.250 | 12.500 | 25.00 |
| Corn Starch | 12.500 | 25.00 | 50.00 |
| Light Magnesium oxide | 0.125 | 0.250 | 0.500 |
| Sodium Lauryl Sulfate | 0.850 | 1.700 | 3.400 |
| Purified water | q.s | q.s | q.s |
| Talc | 0.425 | 0.850 | 1.700 |
| Magnesium stearate | 0.213 | 0.425 | 0.850 |

### Process:

(a) Bumetanide and Pregelatinized Starch were sifted separately.
(b) Lactose monohydrate, Microcrystalline cellulose, corn starch and Lactose monohydrate were sifted.
(c) Sifted Talc separately.
(d) Magnesium stearate was sifted separately.
(e) Loaded the pre-sifted material of step (a) and (b) in a rapid mixer granulator and dry mixed the loaded material.
(f) Dissolved light magnesium oxide in water under stirring followed by addition of Sodium lauryl sulfate under continuous stirring.
(g) Granulated the dry mix material of (e) with solution of (f) and dried the wet mass so obtained.
(h) Sifted the dried granules of step (g).
(i) Blended the granules of step (h) with pre-sifted material of step (c)
(j) Lubricated the blend of step (i) with magnesium stearate of step (d) and compressed the lubricated blend so obtained using suitable punches and dies to form tablets.

Stability Studies: The pharmaceutical composition of the present invention as prepared according to example 6 (0.5 mg, 1.0 mg, 2.0 mg) were tested for stability at 25° C./60% RH and 40° C./75% RH for a period of six months. The amount of nitrosamine impurity is provided in table below:

| | Specification | | | | | |
|---|---|---|---|---|---|---|
| | N-Nitroso | | | | | |
| | bumetanide | | 25° C./60% RH | | 40° C./75% RH | |
| Strength | limit (ppm) | Initial | 3 M | 6 M | 3 M | 6 M |
| 0.5 mg | NMT 2.65 | BDL | BDL | 0.40 | BQL | 0.44 |
| 1.0 mg | | BDL | BDL | BDL | BQL | 0.40 |
| 2.0 mg | | BDL | BDL | 0.40 | BDL | 0.50 |

### Example 7: Pharmaceutical Composition of Bumetanide with Ascorbic Acid

| Ingredient | Quantity % w/w |
|---|---|
| Bumetanide | 0.588 |
| Lactose monohydrate | 61.202 |
| Ascorbic acid | 4.11 |
| Microcrystalline Cellulose | 23.53 |
| Pregelatinized Starch | 8.47 |
| Talc | 1.05 |
| Magnesium stearate | 1.05 |

### Process:

(a) Dispensed and co-sifted Bumetanide, Lactose monohydrate & microcrystalline cellulose.
(b) Prepared solution of pre-gelatinized starch and ascorbic acid in water under continuous stirring.
(c) Granulated the blend of step (a) using solution of step (b)
(d) Lubricated the granules of step (c) using talc and magnesium stearate.
(e) Compressed the blend of step (d) using suitable punches and dies to form tablets.

### Example 8: Pharmaceutical Composition of Propranolol Hydrochloride Extended Release Capsule

| | |
|---|---|
| Ingredient | Quantity (mg) |

Seal Coating

| | | | | |
|---|---|---|---|---|
| Sugar Sphere | 26.250 | 35.00 | 52.50 | 70.00 |
| Hypromellose | 1.181 | 1.575 | 2.362 | 3.150 |
| Talc | 0.131 | 0.175 | 0.263 | 0.350 |
| Purified water | q.s | q.s | q.s | q.s |

Drug Coating

| | | | | |
|---|---|---|---|---|
| Propranolol Hydrochloride | 60.0 | 80.0 | 120.0 | 160.0 |
| Hydroxypropylcellulose | 4.5 | 6.0 | 9.0 | 12.0 |
| Ascorbic acid | 6.0 | 8.0 | 12.0 | 16.0 |
| Talc | 4.5 | 6.0 | 9.0 | 12.0 |
| Purified water | q.s | q.s | q.s | q.s |

Extended Release Coating

| | | | | |
|---|---|---|---|---|
| Ethyl Cellulose | 7.107 | 9.476 | 14.214 | 18.953 |
| Hypromellose | 2.052 | 2.737 | 4.105 | 5.473 |
| Triethyl citrate | 0.071 | 0.094 | 0.142 | 0.189 |
| Isopropyl Alcohol | q.s | q.s | q.s | q.s |
| Methylene Chloride | q.s | q.s | q.s | q.s |

Lubrication

| | | | | |
|---|---|---|---|---|
| Talc | 0.558 | 0.745 | 1.117 | 1.490 |
| Empty Hard Gelatin Capsule | 1 No. | 1 No. | 1 No. | 1 No. |

### Process:

(a) Dissolved Hypromellose in purified water under continuous stirring and added talc into the solution.
(b) Loaded Sugar sphere in FBP and seal coated with solution of step (a).
(c) Added hydroxypropyl cellulose to purified water under continuous stirring followed by addition of ascorbic acid to get homogeneous solution.
(d) Added propranolol hydrochloride to the solution of step (c) under continuous stirring to get homogeneous dispersion.
(e) Added talc to dispersion of step (d) and sifted.
(f) Loaded seal coated sugar sphere of step (b) in FBP and coated the drug to get required weight.
(g) Added Hypromellose to isopropyl alcohol and methylene chloride under continuous stirring followed by addition of Ethyl cellulose, triethyl citrate under continuous stirring
(h) Sifted the solution of step (g).
(i) Coated the drug loaded pellets of step (f) with extended release coating solution of step (h).
(j) Sifted talc and lubricated the coated pellets of step (i) with talc.
(k) Filled the lubricated extended release coated pellets of step (j) in empty hard gelatin capsule using capsule filling machine.

Stability Studies: The pharmaceutical compositions of the present invention as prepared according to example 8 (60 mg, 80 mg, 120 mg, 160 mg) were tested for stability at 25° C./60% RH and 40° C./75% RH for a period of six months. The amount of N-Nitrosopropanolol is provided in table below.

| | |
|---|---|
| 25° C./60% RH | 40° C./75% RH |

| | | | | | |
|---|---|---|---|---|---|
| Strength | Initial | 3 M | 6 M | 3 M | 6 M |

| | | | | | | |
|---|---|---|---|---|---|---|
| 60 | mg | BDL | BDL | BQL | BDL | 0.028 |
| 80 | mg | BDL | BDL | BQL | 0.023 | 0.029 |
| 120 | mg | BDL | BDL | 0.023 | BDL | 0.024 |
| 160 | mg | BDL | BDL | BDL | BQL | 0.023 |

### Example 9: Pharmaceutical Composition of Carbamazepine Immediate Release Tablet

| Ingredient | Quantity (mg) |
|---|---|
| Carbamazepine | 200.00 |
| Microcrystalline cellulose | 95.05 |
| Crospovidone | 2.500 |
| Sodium Lauryl Sulfate | 2.750 |
| Talc | 8.00 |
| Crospovidone | 6.500 |
| Colloidal silicon dioxide | 3.200 |
| Magnesium stearate | 2.00 |

### Process:

(a) Sifted together Carbamazepine, Microcrystalline Cellulose and collected.
(b) Sifted together Crospovidone, Sodium Lauryl Sulfate and collected.
(c) Sifted together mannitol, ascorbic acid and collected.
(d) Sifted talc separately.
(e) Blended the sifted material of step (a) to (d) in non-shear blender and compacted & milled in roller compactor to get granules.
(f) Sifted Microcrystalline Cellulose, Crospovidone and colloidal silicon dioxide together
(g) blended the granules of step (e) with sifted material of step (f).
(h) Compressed the blend of step (g) with pre-sifted magnesium stearate followed by packing the tablets so obtained.
i) Packaged the tablets in a package or container with oxygen scavenger.

The pharmaceutical compositions of the present invention as prepared according to example 9 was compared with the marketed product for the amount of N-Nitroso Iminostilbene and the results are represented as below:

Carbamazepine IR Tablets

| Specification limit | Reference Product | Composition of the |
|---|---|---|
| (ppm) | (Novartis) | invention |
| NMT 0.0166 | 0.227 | 0.0099 |

### Example 10: Pharmaceutical Composition of Carbamazepine Extended Release Capsule

| | | Qty./Capsule (mg) | | |
|---|---|---|---|---|
| S. No. | Ingredient | 100 mg | 200 mg | 300 mg |

Pre compaction mixing

| | | | | |
|---|---|---|---|---|
| 1 | Carbamazepine USP * | 100.000 | 200.000 | 300.000 |
| 2 | Sodium Lauryl Sulphate | 0.667 | 1.333 | 2.000 |
| 3 | Povidone | 8.000 | 16.000 | 24.000 |

Pre compaction lubrication

| | | | | |
|---|---|---|---|---|
| 4 | Talc | 1.000 | 2.000 | 3.000 |

Sustained Release coating

| | | | | |
|---|---|---|---|---|
| 5 | Ammonio methacrylate | 7.440 | 14.880 | 22.320 |
| | copolymer type A | | | |
| 6 | Triethyl citrate | 0.743 | 1.487 | 2.230 |
| 7 | Talc | 3.720 | 7.440 | 11.160 |
| 8 | Purified Water | Qs | Qs | Qs |
| 9 | Acetone | Qs | Qs | Qs |
| 10 | Isopropyl alcohol | Qs | Qs | Qs |

Extra-granular Mixing

| | | | | |
|---|---|---|---|---|
| 11 | Microcrystalline Cellulose | 16.763 | 33.527 | 50.290 |
| 12 | Colloidal Silicon dioxide | 0.667 | 1.333 | 2.000 |

Final lubrication

| | | | | |
|---|---|---|---|---|
| 13 | Talc | 1.000 | 2.000 | 3.000 |

| | | | |
|---|---|---|---|
| Total weight (mg) | 140.000 | 280.000 | 420.000 |

### Brief Manufacturing Process

1. Sifting
1.1 Sifted together carbamazepine, sodium lauryl sulphate and Povidone through suitable sieve and collect in polybag.
1.2 Sifted precompaction Talc through suitable sieve and collect in polybag.
1.3 Sifted Microcrystalline cellulose and colloidal anhydrous silica together through suitable sieve and collect in polybag.
1.4 Sifted final lubrication Talc through suitable sieve and collect in polybag.
2. Blending
2.1 Mixed the step 1.1 materials in suitable non-shear blender for suitable time.
2.2 Lubricated the above blend using sifted precompaction talc of step 1.2 for suitable time.
3. Compaction: Compacted the above blend using roller compactor to achieve the desired granules:fines ratio.
4. Sustained release coating: Performed the coating of the compacted mass (granules) received from step no 3 using Ammonio methacrylate copolymer type A coating composition.
5. Extragranular mixing: Mixed the coated granules of step 4 with step material of step 1.3 in non-shear blender for suitable time.
6. Lubrication: Lubricated the step 5 material using sifted final talc of step 1.4 for suitable time.
7. Capsule filling: Filled the lubricated material of step no 6 in respective capsules sizes strength-wise.
8. Packaged the capsules in a package or container with oxygen scavenger.

The pharmaceutical compositions of the present invention as prepared according to example 10 was compared with the marketed product for the amount of N-Nitroso Iminostilbene and the results are represented as below:

Carbamazepine Extended Release Capsules

| | | Reference | |
|---|---|---|---|
| | Specification limit | Product | Composition according to |
| Strength | (ppm) | (Takeda) | the Present invention |
| 300 mg | NMT 0.0166 | 0.249 | 0.001 |
| 100 mg | | 0.120 | 0.003 |

### Example 11: Pharmaceutical Composition of Carbamazepine Extended Release Tablets

| | | Qty./Tablets (mg) | | |
|---|---|---|---|---|
| S. No. | Ingredient | 100 mg | 200 mg | 400 mg |

Intragranular

| | | | | |
|---|---|---|---|---|
| 1 | Carbamazepine | 100.000 | 200.000 | 400.000 |

Granulation stage I

| | | | | |
|---|---|---|---|---|
| 2 | Ammonio methacrylate copolymer | 10.000 | 20.000 | 40.000 |
| 3 | Dibutyl sebacate | 1.000 | 2.000 | 4.000 |
| 4 | Isopropyl alcohol | Qs | Qs | Qs |
| 5 | Methylene chloride | Qs | Qs | Qs |

Granulation stage II

| | | | | |
|---|---|---|---|---|
| 6 | Hypromellose | 5.000 | 10.000 | 20.000 |
| 7 | Isopropyl alcohol | Qs | Qs | Qs |
| 8 | Methylene chloride | Qs | Qs | Qs |

Granulation stage III

| | | | | |
|---|---|---|---|---|
| 9 | Ethylcellulose | 10.000 | 20.000 | 40.000 |
| 10 | Hypromellose | 1.000 | 2.000 | 4.000 |
| 11 | Dibutyl sebacate | 1.000 | 2.000 | 4.000 |
| 12 | Isopropyl alcohol | Qs | Qs | Qs |
| 13 | Methylene chloride | Qs | Qs | Qs |

Extragranular
Pre-Lubrication Stage

| | | | | |
|---|---|---|---|---|
| 14 | Silicified microcrystaline cellulose | 27.500 | 55.000 | 110.000 |
| 15 | Maize starch | 2.500 | 5.000 | 10.000 |
| 16 | Sodium starch glycolate | 11.250 | 22.500 | 45.000 |

Lubrication Stage

| | | | | |
|---|---|---|---|---|
| 17 | Magnesium stearate | 0.750 | 1.500 | 3.000 |

| | | | |
|---|---|---|---|
| Core Tablet weight (mg) | 170.000 | 340.000 | 680.000 |

### Process:

1. Sifting (Intragranular): Sifted carbamazepine through suitable sieve and collect in polybag.
2. Sifting (Extragranular)
2.1 Sifted together silicified microcrystalline cellulose, maize starch and sodium starch glycolate through suitable sieve and collect in polybag.
2.2 Sifted magnesium stearate through suitable sieve and collect in polybag.
3. Granulation Stage I
3.1 Polymer solution Preparation: Taken methylene chloride in a vessel and added dibutyl sebacate into it under stirring. To above, added Ammonio methacrylate copolymer into it under stirring to make homogenous dispersion. To above, added isopropyl alcohol into it under stirring to make homogenous dispersion.
3.2 Granulation process (Top spray)
   Placed sifted carbamazepine in Fluid bed processor and sprayed the step 3.1 polymer dispersion onto it at desired process parameters and dry the granules till desired % LOD achieved.
4. Granulation Stage II
4.1 Binder solution Preparation
   Taken together Isopropyl alcohol and methylene chloride in a vessel and added Hypromellose into it under stirring to make homogenous dispersion.
4.2 Granulation process (Bottom Spray)
   To the stage I dried granules in Fluid bed processor sprayed the step 4.1 binder dispersion onto it at desired process parameters and dried the granules till desired % LOD achieved.
5. Granulation Stage III
5.1 Polymer solution Preparation
   Taken together Isopropyl alcohol and methylene chloride in a vessel and added dibutyl sebacate into it under stirring. To above, separately added Ethyl cellulose and hypromellose into it under stirring to make homogenous dispersion.
5.2 Granulation process (Bottom spray)
   To the stage II dried granules in Fluid bed processor sprayed the step 5.1 polymer dispersion onto it at desired process parameters. And dry the granules till desired % LOD achieved.
6. Screening of dried granules: Sifted the dried granules through suitable sieve and collect in polybag.
7. Blending: Mixed the step 2.1 of extra-granular materials with step 6 granules in suitable non-shear blender for suitable time. Lubricate the above blend using sifted magnesium stearate of step 2.2 for suitable time.
8. Compression: Compressed the above blend using suitable tooling.
9. Packaged the tablets in a package or container with oxygen scavenger.

The pharmaceutical compositions of the present invention as prepared according to example 11 was compared with the marketed product for the amount of N-Nitroso Iminostilbene and the results are represented as below:

Carbamazepine Extended Release Tablets

| | Specification | Reference Product | Composition according |
|---|---|---|---|
| Strength | limit (ppm) | (Novartis) | to the present invention |
| 400 mg | NMT 0.0166 | 0.020 | 0.015 |
| 200 mg | | 0.029 | 0.016 |
| 100 mg | | 0.017 | 0.035 |

### Example 12: Pharmaceutical Composition of Amitriptyline Hydrochloride Tablets

| Ingredient | Quantity (mg) | | | | | |
|---|---|---|---|---|---|---|
| Amitriptyline | 10.000 | 25.00 | 50.00 | 75.00 | 100.00 | 150.00 |
| Hydrochloride | | | | | | |
| Micro- | 28.417 | 25.000 | 70.108 | 105.162 | 140.216 | 210.324 |
| crystalline | | | | | | |
| Cellulose | | | | | | |
| Hydroxypropyl | 0.823 | 35.054 | 2.166 | 3.249 | 4.332 | 6.498 |
| Cellulose | | | | | | |
| Croscarmellose | 0.823 | 1.083 | 2.166 | 3.249 | 4.332 | 6.498 |
| Sodium | | | | | | |
| Ascorbic acid | 10.000 | 1.083 | 31.062 | 46.593 | 62.124 | 93.186 |
| Isopropyl | qs | qs | qs | qs | qs | qs |
| Alcohol | | | | | | |
| Lactose | 7.278 | 15.531 | 19.166 | 28.749 | 38.332 | 57.498 |
| Monohydrate | | | | | | |
| Croscarmellose | 4.937 | 9.583 | 13.000 | 19.500 | 26.000 | 39.000 |
| Sodium | | | | | | |
| Colloidal | 0.823 | 6.500 | 2.166 | 3.249 | 4.332 | 6.498 |
| Silicon Dioxide | | | | | | |
| Micro- | 6.076 | 8.000 | 16.000 | 24.000 | 32.000 | 48.000 |
| crystalline | | | | | | |
| Cellulose | | | | | | |
| Magnesium | 0.823 | 1.083 | 2.166 | 3.249 | 4.332 | 6.498 |
| Stearate | | | | | | |

Film Coating

### Process:

(a) Blended Amitriptyline HCl, Microcrystalline cellulose, Hydroxypropylcellulose, Ascorbic acid and Croscarmellose Sodium in suitable Rapid Mixer Granulator (RMG)/Blender.
(b) Sifted the blend of step (a) through appropriate sieves
(c) Loaded the blend of Step (b) in RMG and mixed the blend for suitable time.
(d) Granulated the blend of step (c) with Isopropyl alcohol for suitable time.
(e) Kneaded the wet mass obtained in step (d) and discharged the wet granules so obtained.
(f) Air dried the granules of step (e) Fluid Bed Dryer (FBD) for suitable time.
(g) Sifted the granules of step (f) from suitable sieve and suitable mill to get uniform sized wet granules followed by drying in Fluid Bed Dryer (FBD) at 60° C.±10° C. to achieve suitable LOD.
(h) Sifted the granules of step (g) from suitable sieve and milled the oversized granules with suitable mill to get uniform sized dried granules.
(i) Sifted Lactose Monohydrate, Croscarmellose Sodium, Colloidal Silicon dioxide, microcrystalline cellulose through appropriate sieves.
(j) Loaded the Blend of Step (h) granules and Blend of step (i) in suitable blender and blended for suitable time.
(k) Sifted Magnesium stearate through appropriate sieve and lubricated the blend of step
(j) with sifted magnesium stearate in a suitable blender.
(l) Compressed the lubricated blend of step (k) using suitable punches and dies in rotary compression machine.
(m) Dispersed Opadry in a mixture of purified water and Isopropyl alcohol under continuous stirring and stirred for appropriate time and sifted the coating dispersion
(n) Film coated the compressed tablets of step (1) with coating of step (m).

The pharmaceutical compositions of the present invention as prepared according to example 11 (10 mg, 25 mg, 50 mg, 75 mg, 100 mg, 150 mg) were tested. The amount of N-Nitrosonortriptyline impurity is provided in table below.

| | | |
|---|---|---|
| | Specification limit | Composition of the |
| Strength | (ppm) | present invention |

| | | | |
|---|---|---|---|
| 10 | mg | NMT 0.088 ppm | 0.010 |
| 25 | mg | | 0.007 |
| 50 | mg | | 0.007 |
| 75 | mg | | 0.008 |
| 100 | mg | | 0.008 |
| 150 | mg | | 0.007 |

Wherever applicable:
Specification limit corresponds to FDA acceptable limit based on maximum daily dose of therapeutically active agent.
BDL is Below Detection Limit
BQL is Below Quantification Limit
NMT is Not more than

1. A stable pharmaceutical composition comprising a therapeutically active agent, one or more stabilizing agents, and one or more pharmaceutically acceptable excipients.

2. The stable pharmaceutical composition according to claim 1, wherein the amount of nitrosamine impurity after exposure of the pharmaceutical composition to 40° C./75% RH for a period of six months is less than about 50 ppm.

3-5. (canceled)

6. The stable pharmaceutical composition according to claim 1, wherein the stabilizing agent is selected from the group comprising antioxidants, amino acids, sugar alcohols, carbohydrates, pH modifiers, or a combination thereof.

7-12. (canceled)

13. The stable pharmaceutical composition according to claim 1, wherein the ratio of the therapeutically active agent to the stabilizing agent is 1:0.01 to 1:25.

14. (canceled)

15. The stable pharmaceutical composition according to claim 6, wherein the antioxidant is selected from ascorbic acid or butylated hydroxytoluene, butylated hydroxyanisole, or a combination thereof.

16. (canceled)

17. The stable pharmaceutical composition according to claim 6, wherein the pH modifier is selected from magnesium oxide, sodium carbonate, sodium bicarbonate, potassium bicarbonate, lithium carbonate, potassium carbonate, calcium carbonate, magnesium carbonate, sodium citrate, sodium hydroxide, potassium hydroxide, ammonium salts or a combination thereof.

18. The stable pharmaceutical composition according to claim 46, wherein the composition further comprises:
10% w/w-80% w/w of diluent,
0.1% w/w-10% w/w of disintegrant, and
0.1% w/w-5% w/w of lubricant; based on the total weight of the composition.

19-21. (canceled)

22. The stable pharmaceutical composition according to claim 1, wherein the therapeutically active agent is a drug having secondary, tertiary amine, or quaternary amine group susceptible to formation of nitrosamine impurity.

23. The stable pharmaceutical composition according to claim 1, wherein the therapeutically active agent is sitagliptin, empagliflozin, dapagliflozin, varenicline, nebivolol, verapamil, metoprolol, carvedilol, diltiazem, esomeprazole, pantoprazole, telmisartan, olmesartan, divalproex, haloperidol, ezetemibe, fenofibrate, atorvastatin, baclofen, succinylcholine, sugammadex, timolol, tobramycin, carbidopa, levodopa, phenytoin, sacubitril, nitrofurantoin, rivaroxaban, loteprednol ticagrelor mesalamine, arformoterol, vasopressin, phenylephrine, lidocaine, brimonidine, bimatoprost, tobramycin, brinzolamide, dorzolamide, tetracaine, ephedrine, epinephrine norepinephrine, moxifloxacin, ciprofloxacin, revefenacin, rocuronium, latanoprost, lifitegrast, methylprednisolone, glycopyrrolate, apremilast, metformin, valsartan, propranolol, bumetanide, carbamazepine, or amitriptyline.

24. A package or a storage container for packaging or storing the stable pharmaceutical composition according to claim 1, wherein the package or container is made up of material that blocks oxygen or is impervious to oxygen.

25. A package or a storage container for packaging or storing the stable pharmaceutical composition according to claim 1, wherein the package or container comprises an oxygen scavenger.

26-45. (canceled)

46. The stable pharmaceutical composition according to claim 1, wherein the one or more pharmaceutically acceptable excipients are selected from the group comprising diluents, disintegrants, glidants, lubricants, surface active agents, buffers, isotonicity agents, viscosity enhancers, pH adjusting agents, preservatives, vehicles, bulking agents and lyoprotectants and/or combinations thereof.
